(19) United States
(12) Patent Application Publication
(10) Pub. No.: US 2020/0365255 A1
(43) Pub. Date: Nov. 19, 2020

(54) MEDICAL INFORMATION PROCESSING DEVICE, MEDICAL INFORMATION PROCESSING METHOD, AND STORAGE MEDIUM

(71) Applicant: NEC Corporation, Tokyo (JP)

(72) Inventors: Masahiro HAYASHITANI, Tokyo (JP); Yutaka TSUBOISHI, Tokyo (JP); Shuuya MAEDA, Tokyo (JP); Masahiro KUBO, Tokyo (JP)

(73) Assignee: NEC Corporation, Tokyo (JP)

(21) Appl. No.: 16/638,168

(22) PCT Filed: Aug. 23, 2018
(86) PCT No.: PCT/JP2018/031096

§ 371 Date: Feb. 11, 2020

(30) Foreign Application Priority Data
Aug. 30, 2017 (JP) 2017-165378

(51) Int. Cl. G16H 40/20; G16H 50/20; G16H 50/30

(52) U.S. Cl. CPC G16H 40/20; G16H 50/30; G16H 50/20

(57) ABSTRACT

The present invention assists a medical practitioner in making a highly reliable determination of a discharge destination. The medical information processing device according to the present invention is provided with: an acquiring unit for acquiring a discharge destination prediction result that is the result of predicting the discharge destination, and a model related to the discharge destination prediction result, the model being for classifying the discharge destination using a data item included in an electronic medical record as an explanatory variable; an extraction unit for extracting, from the model, a data item that affects the discharge destination prediction and that satisfies a predetermined condition; and an output unit for correlating and outputting the data item with the discharge destination prediction result on the basis of the extraction result.

### TECHNICAL FIELD

The present disclosure relates to a medical information processing device, a medical information processing method, and a program.

### BACKGROUND ART

A technique for a prognosis prediction of a patient is disclosed (PTLs 1 to 3). PTL 1, for example, describes that a notification indicating a prediction of prognosis of a patient is presented in association with current physiological information and a medical history record of a patient.

Further, PTL 2 describes a system that calculates a risk of death by using a prediction model related to a risk of death after hospital discharge, calculates a risk of rehospitalization by using the prediction model related to a risk of rehospitalization after hospital discharge, and presents a risk on hospital discharge to a doctor, based on these risks.

Further, PTL 3 describes that setting of an adaptive recovery environment is determined, based on a patient parameter set being information relating to a patient and being determined by using machine learning.

Note that, as one example of a prediction using a prediction model, a technique for predicting an energy demand is described in PTL 4.

### CITATION LIST

### Patent Literature

[PTL 1] Japanese Unexamined Patent Application Publication No. 2012-221508
[PTL 2] Japanese Translation of PCT International Application Publication No. 2014-520335
[PTL 3] Japanese Translation of PCT International Application Publication No. 2016-532459
[PTL 4] Japanese Unexamined Patent Application Publication No.

### SUMMARY OF INVENTION

### Technical Problem

However, in the techniques described in the patent literatures described above, it is unclear by what piece of information relating to a patient an output prognosis prediction is predicted. Therefore, when a medical practitioner such as a doctor predicts a discharge destination of a patient by using an output prognosis prediction, it may be difficult to determine, depending on information relating to a patient, a discharge destination suitable for the patient.

Therefore, reliability for a discharge destination determined by a medical practitioner may decrease.

The present disclosure has been made in view of the above problem and an object thereof is to provide a technique for assisting a medical practitioner in determining a highly reliable discharge destination.

### Solution to Problem

A medical information processing device according to an example aspect of the present disclosure includes acquiring means for acquiring a discharge destination prediction result being a result of predicting a discharge destination and a model that is related to the discharge destination prediction result and is used for classifying the discharge destination by using a data item included in an electronic medical record as an explanatory variable, extraction means for extracting, from the model, the data item that affects a prediction of the discharge destination and satisfies a predetermined condition and output means for outputting, based on an extraction result, the data item in association with the discharge destination prediction result.

Further, a medical information processing method according to an example aspect of the present disclosure includes acquiring a discharge destination prediction result being a result of predicting a discharge destination and a model that is related to the discharge destination prediction result and is used for classifying the discharge destination by using a data item included in an electronic medical record as an explanatory variable, extracting, from the model, the data item that affects a prediction of the discharge destination and satisfies a predetermined condition and outputting, based on an extraction result, the data item in association with the discharge destination prediction result.

A computer program that achieves the devices or the method described above by a computer, and a computer-readable non-transitory recording medium storing the computer program are also included in the scope of the present disclosure.

### Advantageous Effects of Invention

The present disclosure is able to suitably assist a medical practitioner in determining a highly reliable discharge destination.

### BRIEF DESCRIPTION OF DRAWINGS

FIG. 1 is a block diagram illustrating one example of a configuration of a medical information processing device according to a first example embodiment.

FIG. 2 is a flowchart illustrating one example of a flow of processing of the medical information processing device according to the first example embodiment.

FIG. 3 is a block diagram illustrating one example of a configuration of a medical information processing device according to a second example embodiment.

FIG. 4 is a diagram illustrating one example of model information.

FIG. 5 is a diagram illustrating one example of an output screen.

FIG. 6 is a flowchart illustrating one example of a flow of processing of the medical information processing device according to the second example embodiment.

FIG. 7 is a diagram illustrating another example of the output screen.

FIG. 8 is a diagram illustrating another example of the output screen.

FIG. 9 is a diagram illustrating another example of the model information.

FIG. 10 is a diagram illustrating another example of the output screen.

FIG. 11 is a diagram illustrating another example of the model information.

FIG. 12 is a diagram illustrating another example of the model information.

FIG. 13 is a diagram illustrating another example of the model information.

FIG. 14 is a diagram illustrating another example of the model information.

FIG. 15 is a diagram illustrating another example of the output screen.

FIG. 16 is a block diagram illustrating one example of a configuration of a medical information processing device according to a third example embodiment.

FIG. 17 is a diagram illustrating one example of condition information.

FIG. 18 is a flowchart illustrating one example of a flow of processing of the medical information processing device according to the third example embodiment.

FIG. 19 is a diagram exemplarily illustrating a hardware configuration of a computer (information processing device) capable of achieving the example embodiments.

### EXAMPLE EMBODIMENT

### First Example Embodiment

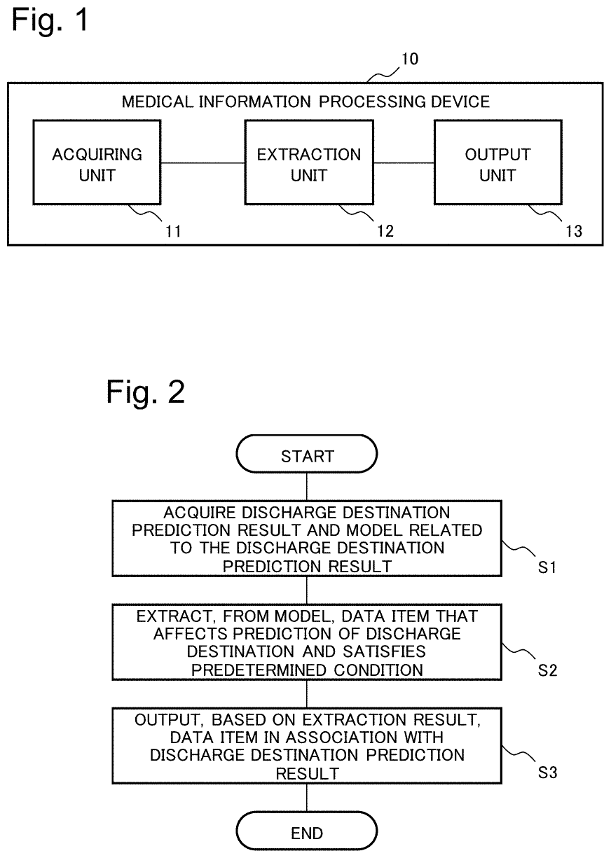

FIG. 1 is a block diagram illustrating one example of a configuration of a medical information processing device 10 according to the present example embodiment. As illustrated in FIG. 1, the medical information processing device 10 according to the present example embodiment includes an acquiring unit 11, an extraction unit 12, and an output unit 13.

The acquiring unit 11 acquires a discharge destination prediction result that is a result of predicting a discharge destination and a model that is related to the discharge destination prediction result and is used for classifying the discharge destination. The model uses a data item included in an electronic medical record as an explanatory variable. The data item included in the electronic medical record includes personal information (attribute information) of a patient such as a gender, an age, and an address, and information relating to a state of a patient such as a consciousness level (a presence or absence of consciousness and a degree thereof).

The discharge destination indicates a place of a destination moved from a place (e.g., a hospital to which emergency transport is carried out) where a patient is hospitalized, and includes, but not limited to, for example, a home, a medical treatment hospital or hospital ward, a hospital or hospital ward for rehabilitation, a nursing-care facility, and the like.

The model is previously generated and stored in any storage unit. A place where the model is stored may be in the medical information processing device 10 or may be in a storage device separate from the medical information processing device 10. The model may be generated, for example, by performing any machine learning or the like by using an electronic medical record of a patient as learning data. The model may be generated, for example, by heterogeneous mixture learning. The model may be, for example, a model for performing two-class classification in order of severity.

The discharge destination prediction result is a result of predicting a discharge destination of a patient by using any prediction method. The prediction method of a discharge destination may be a method of performing a prediction by using the model or may be another method. The discharge destination prediction result includes at least attribute information (a patient identifier and a patient name) of a patient and a place (a home, a hospital, a hospital ward, a facility, and the like) predicted as a discharge destination of the patient.

The acquiring unit 11 acquires, based on the acquired discharge destination prediction result, a model related to the discharge destination prediction result. The model acquired by the acquiring unit 11 may be a model related to the discharge destination prediction result. When, for example, a prediction of a discharge destination is performed by using a model, the acquiring unit 11 acquires the model. When, for example, it is unclear by what method a prediction of a discharge destination is performed, the acquiring unit 11 may estimate a used model from the discharge destination prediction result and acquire the estimated model. Note that the acquiring unit 11 may acquire one model or a plurality of models. When the acquiring unit 11 estimates a model, the acquiring unit 11 may estimate the model, for example, based on a value (item value) of each data item of an electronic medical record or may estimate the model by using another method. Further, the acquiring unit 11 may acquire, when performing a prediction, for example, by using a model, a predetermined number of models among a plurality of the models in which a discharge destination indicated by the discharge destination prediction result can be a prediction result. When the model is, for example, a model used for determining whether one discharge destination is output as a prediction result, the acquiring unit 11 may acquire a predetermined number of the models used for determining whether a discharge destination included in a discharge destination prediction result is output as a prediction result.

The extraction unit 12 extracts, from the model, a data item that affects the prediction of the discharge destination and satisfies a predetermined condition. The extraction unit 12 extracts, for example, a data item having a positive sign of a coefficient (also referred to as related information) associated with a data item of an electronic medical record included in the model as an explanatory variable, and a data item having a negative sign of the coefficient. The extraction unit 12 may, for example, sum, for each data item and each sign, coefficients associated with data items included in each of a plurality of models and extract, in descending order of a summed value, a predetermined number of data items related to the summed value.

The output unit 13 outputs, based on the extraction result acquired by the extraction unit 12, the data item extracted by the extraction unit 12 in association with the discharge destination prediction result. The output unit 13 outputs, for example, the data item and the discharge destination prediction result to a display device, and thus displays the data item and the discharge destination prediction result on a screen of the display device. Further, the output unit 13 outputs, for example, the data item and the discharge destination prediction result to a printing device such as a printer, and thus prints the data item and the discharge destination prediction result on printing paper.

FIG. 2 is a flowchart illustrating one example of a flow of processing of the medical information processing device 10 according to the present example embodiment. As illustrated in FIG. 2, the acquiring unit 11 in the medical information processing device 10 acquires a discharge destination prediction result that is a result of predicting a discharge destination and a model that is related to the discharge destination prediction result and uses a data item included in an electronic medical record as an explanatory variable (step S1).

The extraction unit 12 extracts, from the model acquired in step S1, a data item that affects the prediction of the discharge destination and satisfies a predetermined condition (step S2).

The output unit 13 outputs, based on the extraction result, the data item in association with the discharge destination prediction result (step S3).

As described above, processing of the medical information processing device 10 is terminated.

As described above, the medical information processing device 10 according to the present example embodiment outputs a discharge destination prediction result and a data item of an electronic medical record extracted from a model related to the discharge destination prediction result in association with each other. The model uses the data item included in the electronic medical record as an explanatory variable. Therefore, a medical practitioner who confirms a discharge destination prediction result can easily understand that a data item associated with the discharge destination prediction result is the data item of the electronic medical record that affects the discharge destination prediction result.

Thereby, the medical information processing device 10 suitably assists a medical practitioner in determining whether a discharge destination indicated by a discharge destination prediction result is suitable as a discharge destination of a patient. When, for example, the data item that affects the discharge destination prediction result is the same as a data item to be referred to by a medical practitioner when determined as a discharge destination of a patient, a medical practitioner can determine that reliability of the discharge destination prediction result is high.

Further, when, for example, the data item that affects the discharge destination prediction result is different from a data item to be referred to by a medical practitioner when determined as a discharge destination of a patient, a medical practitioner can study whether a discharge destination indicated by the discharge destination prediction result is a suitable discharge destination, based on a content input to an electronic medical record, a history of an input content, a conversation with another medical practitioner, and the like. Therefore, a medical practitioner can determine, based on the study result, a highly reliable discharge destination for the patient. Therefore, the medical information processing device 10 according to the present example embodiment can suitably assist a medical practitioner in determining a highly reliable discharge destination.

Further, according to the present disclosure, a medical practitioner determines a highly reliable discharge destination, whereby contribution can be made to prevention of rehospitalization of a patient.

### Second Example Embodiment

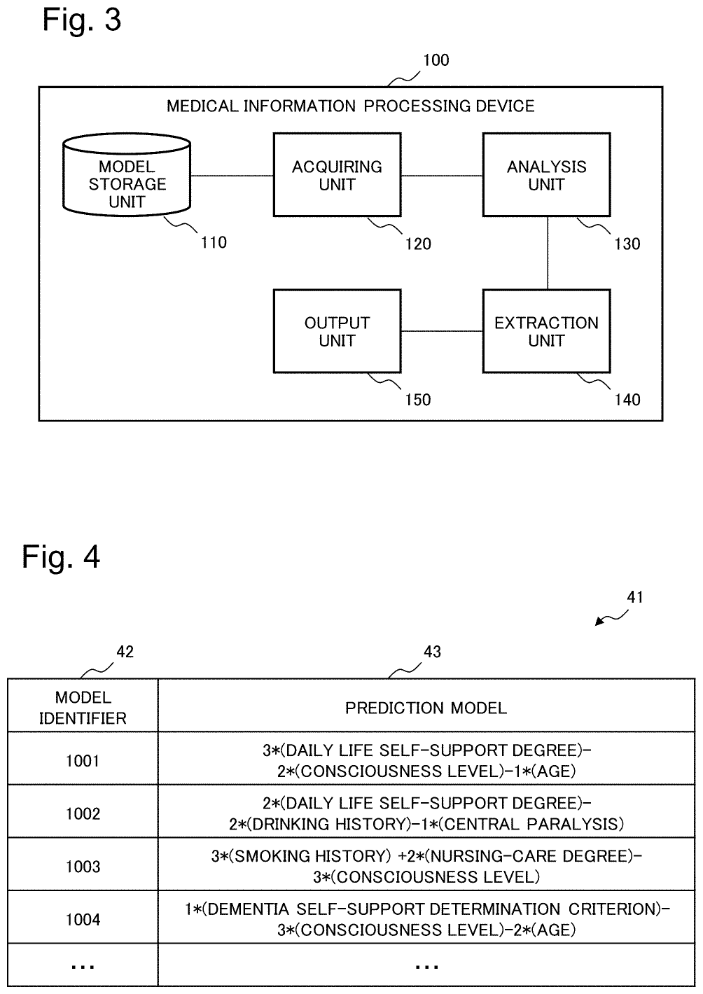

FIG. 3 is a block diagram illustrating one example of a configuration of a medical information processing device 100 according to the present example embodiment. The medical information processing device 100 includes a configuration common to the medical information processing device 10 according to the first example embodiment. Specifically, the medical information processing device 100 includes a model storage unit 110, an acquiring unit 120, an analysis unit 130, an extraction unit 140, and an output unit 150.

A term used also according to the first example embodiment among terms to be used according to the following example embodiment and modified examples is used with the same meaning as a term used according to the first example embodiment unless otherwise noted. Further, according to the present example embodiment, a component assigned with the same number as a component described according to the first example embodiment is a component similar to a component having the same number described according to the first example embodiment. Therefore, overlapping description of such a component may be appropriately omitted.

The model storage unit 110 stores model information including a model (referred to as a prediction model) for classifying a discharge destination. The prediction model may be used for predicting a discharge destination. FIG. 4 is a diagram illustrating one example of the model information stored in the model storage unit 110. The model storage unit 110 stores model information 41, for example, as illustrated in FIG. 4. The model information 41 includes a model identifier 42 that identifies each prediction model and a prediction model 43. Note that the model information 41 may include information other than information illustrated in FIG. 4.

The prediction model 43 is a previously generated model. The prediction model 43 may be generated, for example, by performing any machine learning or the like. The prediction model 43 may be generated, for example, by heterogeneous mixture learning.

The prediction model 43 is expressed in a format of a polynomial. Note that an expression method of the prediction model 43 is not specifically limited. Each term of the prediction model 43 includes a variable (explanatory variable) and a coefficient (related information). In FIG. 4, the variable is a portion surrounded by parentheses. The variable is a data item (also referred to simply as an item) of an electronic medical record. The item of the electronic medical record includes personal information (attribute information) of a patient such as a gender, an age, and an address, and information relating to a state of a patient such as a consciousness level (a presence or absence of consciousness and a degree thereof). A value substituted into the variable is a value related to the item. The value (a numerical value and information) related to the item may be input by a medical practitioner such as a doctor and a nurse, at a time of hospital admission of a patient or may be input and updated by a medical practitioner after hospital admission. Note that, according to the present example embodiment, description is made, assuming that the value related to the item is digitized, but, without limitation thereto, such a value may not necessarily digitized. In this case, when a prediction is performed by using the prediction model 43, the value related to the item may be converted to a numerical value. The item used as the variable of the prediction model 43 among items of an electronic medical record and a magnitude of the coefficient associated (multiplied) with the variable are determined by machine learning. Note that the variable of the prediction model 43 may be a factor that affects, when predicting a discharge destination by using the prediction model 43, the prediction of the discharge destination. Therefore, the variable of the prediction model 43 is also referred as a factor.

A result (numerical value) calculated by using the prediction model 43 can be a prediction result of the discharge destination. Each prediction model 43 may be able to predict one discharge destination from a plurality of discharge destinations, based on the calculated numerical value. Further, each prediction model 43 may determine whether one discharge destination is output as a prediction result.

The acquiring unit 120 is one example of the acquiring unit 11 according to the first example embodiment. The acquiring unit 120 acquires a discharge destination prediction result input from an outside of the medical information processing device 100 or acquired by predicting a discharge destination in an inside. The discharge destination prediction result includes at least attribute information (a patient identifier and a patient name) of a patient and a place (a home, a hospital, a hospital ward, and a facility) predicted as a discharge destination of the patient. A prediction method of the discharge destination for generating the discharge destination prediction result acquired by the acquiring unit 120 is not specifically limited and any prediction method is employable. The prediction method of the discharge destination may be a method of performing a prediction using the model information 41 or another method.

The acquiring unit 120 acquires, based on the acquired discharge destination prediction result, a prediction model 43 related to the discharge destination prediction result from the model storage unit 110. When the discharge destination prediction result includes information (a model identifier 42) indicating a used prediction model 43, the acquiring unit 120 acquires the prediction model 43 associated with the model identifier 42. When the discharge destination prediction result does not include the model identifier 42, the acquiring unit 120 estimates a used prediction model 43 from the discharge destination prediction result and acquires the estimated prediction model 43. Note that the acquiring unit 120 may acquire one prediction model 43 or a plurality of prediction models 43. An estimation method of the prediction model 43 by the acquiring unit 120 is not specifically limited and an estimation may be performed, for example, based on an item value of each item of an electronic medical record. Further, the acquiring unit 120 may acquire, when performing a prediction, for example, by using a prediction model 43, a predetermined number of prediction models 43 among prediction models 43 in which a discharge destination indicated by the discharge destination prediction result can be a prediction result. When the prediction model 43 is, for example, a model used for determining whether one discharge destination is output as a prediction result, the acquiring unit 120 may acquire a predetermined number of the prediction models 43 used for determining whether a discharge destination included in a discharge destination prediction result is output as a prediction result.

The acquiring unit 120 supplies the acquired prediction model 43 to the analysis unit 130, together with the discharge destination prediction result.

The analysis unit 130 and the extraction unit 140 are one example of the extraction unit 12 according to the first example embodiment described above. The analysis unit 130 analyzes the prediction model 43 acquired by the acquiring unit 120. Specifically, the analysis unit 130 identifies, from the prediction model 43, an item (explanatory variable) of an electronic medical record that affects the discharge destination prediction result. At that time, the analysis unit 130 preferably extracts, together with the extracted item, a coefficient (related information) associated with the item. When there are a plurality of the prediction models 43, the analysis unit 130 may calculate a total value of the coefficients with respect to each item. This total value is associated with the item of the electronic medical record that affects the discharge destination prediction result, and therefore it can be said that the total value represents a degree of affecting the discharge destination prediction result.

The analysis unit 130 preferably identifies, based on a sign, an item (first item) to be estimated as contributing in such a way as to be predicted as the discharge destination indicated by the discharge destination prediction result and an item (second item) estimated as contributing in such a way as not to be predicted as the discharge destination indicated by the discharge destination prediction result. The second item is an item contributing in such a way as not to be predicted as the discharge destination indicated by the discharge destination prediction result and therefore can be referred to as an item contributing in such a way as to be predicted as another discharge destination different from the discharge destination indicated by the discharge destination prediction result. The analysis unit 130 identifies, when a sign of the coefficient associated with the item is positive, the item as the first item and identifies, when a sign of the coefficient associated with the item is negative, the item as the second item.

The analysis unit 130 may calculate, by using the coefficient associated with the first item, a total value being a degree of contributing (affecting) in such a way as to be predicted as the discharge destination and calculate, by using the coefficient associated with the second item, a total value being a degree of affecting in such a way as not to be predicted as the discharge destination.

The analysis unit 130 supplies, as an analysis result, the identified item and the total value of coefficients being the degree of affecting the discharge destination prediction result with respect to each item to the extraction unit 140, together with the discharge destination prediction result. According to the present example embodiment, description is made, assuming that a total value of coefficients is calculated for each item and each sign.

The extraction unit 140 extracts an item that satisfies a predetermined condition, based on the analysis result. The extinction unit 140 extracts, based on the analysis result, for example, a predetermined number of items in descending order of the degree of affecting the discharge destination prediction result. The extraction unit 140 supplies the extracted item and the total value to the output unit 150 as an extraction result, together with the discharge destination prediction result.

The operations of the analysis unit 130 and the extraction unit 140 are further described with reference to FIG. 4. It is assumed that, for example, the discharge destination prediction result acquired by the acquiring unit 120 indicates a “home” and the acquiring unit 120 acquires, from the model storage unit 110, prediction models 43 in which the model identifier 42 of FIG. 4 is associated with each of “1001”, “1002”, and “1004”.

The analysis unit 130 identifies, among items of an electronic medical record, the items included in the models as the items of the electronic medical record that affect the discharge destination prediction result. In other words, the analysis unit 130 identifies a “daily life self-support degree”, a “consciousness level”, an “age”, a “drinking history”, “central paralysis”, and a “dementia self-support determination criterion” as the items of the electronic medical record that affect the discharge destination prediction result.

Then, the analysis unit 130 calculates a total value of coefficients for each item and each sign of the coefficients. Therefore, a total value of the coefficients having a positive sign is, for each item, 5 as the “daily life self-support degree” and 1 as the “dementia self-support determination criterion”. A total value of the coefficients having a negative sign is, for each item, −5 as the “consciousness level”, −3 as the “age”, −2 as the “drinking history”, and −1 as “central paralysis”.

When, for example, the predetermined number is 2, the extraction unit 140 extracts, from the prediction models 43, the “daily life self-support degree” and the “dementia self-support determination criterion” that are two items in descending order of the total value of the coefficients having the positive sign. Further, the extraction unit 140 extracts, from the prediction models 43, the “consciousness level” and the “age” that are two items in ascending order of the total value of the coefficients having the negative sign (in descending order in a negative direction or in descending order of an absolute value of the total value of the coefficients). In this manner, the analysis unit 130 and the extraction unit 140 extract, from the model, a data item that satisfies a predetermined condition, based on a sign of the coefficient.

The extraction unit 140 supplies, as an extraction result, the extracted “daily life self-support degree”, “dementia self-support determination criterion”, “consciousness level”, and “age” to the output unit 150, together with each total value.

The output unit 150 is one example of the output unit 13 according to the first example embodiment. The analysis unit 130 outputs, based on the extraction result by the extraction unit 140, the item extracted by the extraction unit 140 in association with the discharge destination prediction result. The output unit 150 outputs the item and the discharge destination prediction result to a display device and displays the item and the discharge destination prediction result on a screen of the display device. Further, the output unit 150 outputs the item and the discharge destination prediction result, for example, to a printing device such as a printer and prints the item and the discharge destination prediction result on printing paper. A destination to which the output unit 150 outputs the item and the discharge destination prediction result is not specifically limited and may be, for example, a storage device or another device.

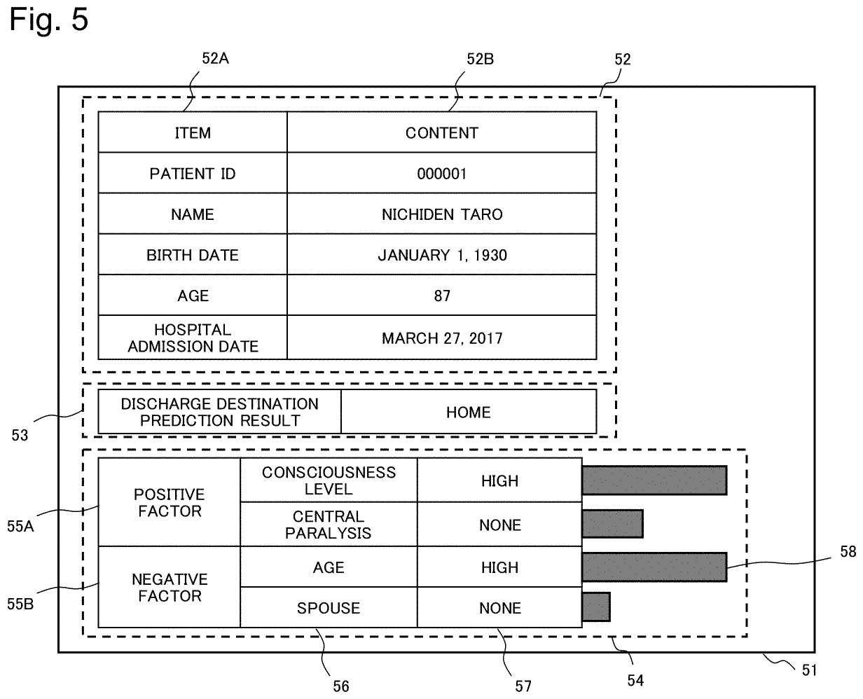

FIG. 5 is a diagram illustrating one example of an output screen to be displayed by a display device when the output unit 150 executes output to the display device. Also, in the following, description is made, assuming that an output destination of the output unit 150 is a display device. The output screen illustrated in FIG. 5 is a display screen using an extraction result different from the extraction result specifically described by using FIG. 4. Note that a display form of the output screen displayed by the display device is one example and is not limited thereto.

An output screen 51 illustrated in FIG. 5 includes an area 52 that displays attribute information of a patient, an area 53 that displays a discharge destination prediction result, and an area 54 that displays information relating to the item extracted by the extraction unit 140.

The area 52 includes an item 52A associated with an item of an electronic medical record and a content 52B associated with an item value of the item of the electronic medical record. Attribute information of a patient to be displayed in the area 52 may include information that identifies a patient.

The area 53 displays a discharge destination indicated by a discharge destination prediction result.

The area 54 displays information relating to the item extracted by the extraction unit 140. The item is associated with the discharge destination prediction result displayed in the area 53. The area 54 includes a positive factor 55A, a negative factor 55B, an item 56, an item value 57, and a parameter 58.

The positive factor 55A is information (first information) indicating that contribution is made in such a way as to be predicted as the discharge destination indicated by the discharge destination prediction result. The negative factor 55B is information (second information different from the first information) indicating that contribution is made in such a way as not to be predicted as the discharge destination indicated by the discharge destination prediction result.

The item 56 is an item of the electronic medical record extracted by the extraction unit 140 from a prediction model 43. The item 56 associated with the positive factor 55A is an item (first item) that contributes in such a way as to be predicted as the discharge destination indicated by the discharge destination prediction result. The item 56 associated with the negative factor 55B is an item (second item) that contributes in such a way as not to be predicted as the discharge destination indicated by the discharge destination prediction result (or in such a way as to be predicted as another discharge destination different from the discharge destination indicated by the discharge destination prediction result).

In this manner, the output unit 150 outputs the first item and first information (the positive factor 55A) in association with each other and outputs the second item in association with the second information (the negative factor 55B) different from the first information. In other words, the output unit 150 outputs, for example, to the display device or the like, whether the item 56 that affects the discharge destination prediction result contributes to a direction of being predicted as the discharge destination indicated by the discharge destination prediction result or contributes another direction in a form understandable by a medical practitioner. Thereby, the medical information processing device 100 can cause a medical practitioner to easily understand a direction to which the item 56 contributes.

The item value 57 is a value of the item 56 and is associated with the item 56. The item value 57 may be an item value itself of the item of the electronic medical record or may be a value acquired from a result of comparing the item value with a predetermined criterion. For example, the item value 57 associated with the item of the “age” among item values 57 illustrated in FIG. 5 is “high” being a result of comparison of whether to be higher than a predetermined criterion.

The output unit 150 outputs, in this manner, the item 56 associated with the discharge destination prediction result in association with the item value 57 and thereby can cause a medical practitioner to easily understand the item value 57 of the item 56 that affects the discharge destination prediction result.

The parameter 58 indicates, by using a graph, a total value of the coefficients (related information) associated with the item 56. A display form of the parameter 58 may be a numerical value representing a total value instead of a graph or may be expressed by a shape and a color. In this manner, the output unit 150 outputs the total value of the coefficients being the parameter 58 in association with the item 56 and thereby can cause a medical practitioner to easily understand a degree in which the item 56 that affects the discharge destination prediction result contributes (affects) in such a way as to be predicted as the discharge destination.

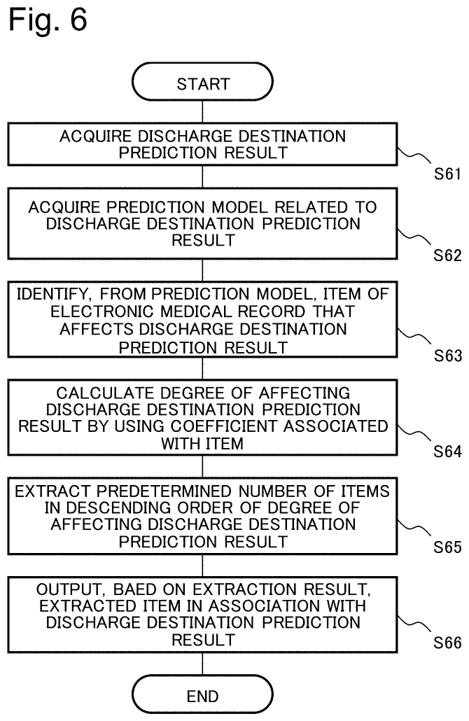

FIG. 6 is a flowchart illustrating one example of a flow of processing of the medical information processing device 100 according to the present example embodiment. As illustrated in FIG. 6, the acquiring unit 120 in the medical information processing device 100 acquires a discharge destination prediction result being a result of predicting a discharge destination predicted in an external device or an inside of the medical information processing device 100 (step S61).

Next, a prediction model 43 related to the discharge destination prediction result acquired by the acquiring unit 120 is acquired from the model storage unit 110 (step S62).

The analysis unit 130 identifies, from the prediction model 43, an item of an electronic medical record that affects the discharge destination prediction result (step S63). The analysis unit 130 calculates, by using a coefficient associated with the item, a degree (a total value of the coefficients) in which the identified item affects the discharge destination prediction result, by using a coefficient associated with the item. In other words, the analysis unit 130 calculates the total value of the coefficients with respect to each item and each sign of the coefficients (step S64).

The extraction unit 140 extracts a predetermined number of the items in descending order of the degree of affecting the discharge destination prediction result (step S65). Specifically, the extraction unit 140 extracts a predetermined number of the items in descending order of a total value with respect to each item of a coefficient when a sign of the coefficient is positive and extracts a predetermined number of the items in ascending order of a total value with respect to each item of a coefficient when a sign of the coefficient is negative (in descending order of an absolute value of the total value of the coefficients).

The output unit 150 outputs, based on the extraction result, the extracted item in association with the discharge destination prediction result (step S66). As described above, the medical information processing device 100 terminates the processing.

The medical information processing device 100 according to the present example embodiment outputs a discharge destination prediction result and an item of an electronic medical record extracted from a prediction model 43 related to the discharge destination prediction result in association with each other. Therefore, a medical practitioner who confirms a discharge destination prediction result can easily understand that an item associated with the discharge destination prediction result is the item of the electronic medical record that affects the discharge destination prediction result.

Thereby, the medical information processing device 100 can suitably assist a medical practitioner in determining whether a discharge destination indicated by a discharge destination prediction result is suitable as a discharge destination of a patient. When, for example, the item that affects the discharge destination prediction result is the same as an item to be referred to by a medical practitioner when determined as a discharge destination of a patient, a medical practitioner can determine that reliability of the discharge destination prediction result is high.

Further, when, for example, the item that affects the discharge destination prediction result is different from as an item to be referred to by a medical practitioner when determined as a discharge destination of a patient, a medical practitioner can study whether a discharge destination indicated by the discharge destination prediction result is a suitable discharge destination, based on a content input to an electronic medical record, a history of an input content, a conversation with another medical practitioner, and the like. Therefore, a medical practitioner can determine, based on the study result, a highly reliable discharge destination for the patient. Therefore, the medical information processing device 100 according to the present example embodiment can suitably assist a medical practitioner in determining a highly reliable discharge destination.

Note that a discharge destination indicated by a discharge destination prediction result may be a specific place name or may be a type of a discharge destination. When, for example, a discharge destination is a hospital, the discharge destination may be information indicating a type of a hospital (e.g., a rehabilitation hospital, a medical treatment hospital, and a nursing-care hospital). When, for example, a discharge destination is a facility different from a hospital, the discharge destination may be information indicating a type of a facility (e.g., a long-term care health facility, a facility covered by public aid providing long-term care to the elderly, and a fee-based home for the elderly).

### Modified Example 1

A discharge destination indicated by a discharge destination prediction result acquired by the acquiring unit 120 is not limited to one discharge destination. The discharge destination prediction result may include a plurality of discharge destinations associated with a probability of a prediction. In this case, the analysis unit 130 and the extraction unit 140 execute an operation similar to the above-described operation for each discharge destination and supply, as an extraction result, an item extracted with respect to each of the plurality of discharge destinations included in the discharge destination prediction result to the output unit 150, together with the discharge destination prediction result.

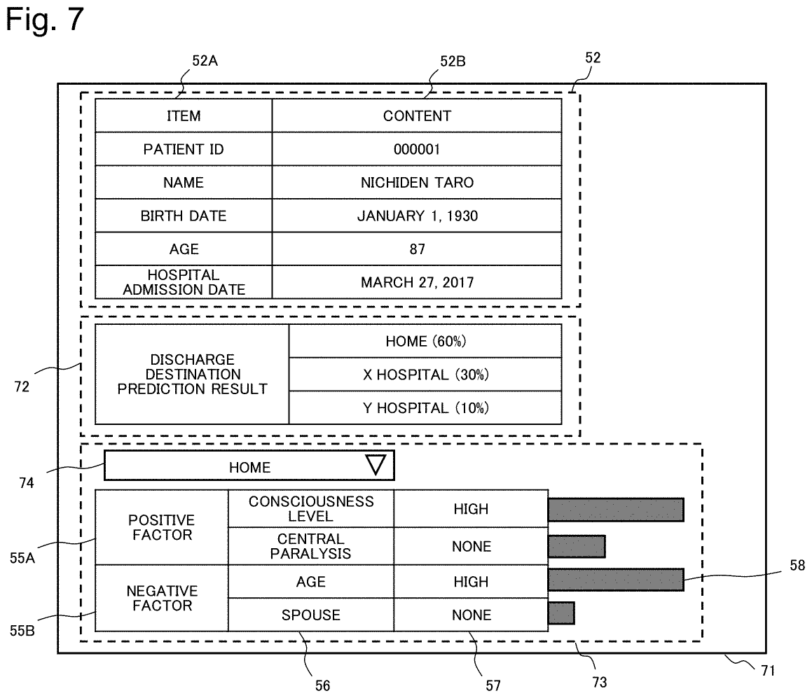

FIG. 7 is a diagram illustrating one example of an output screen output to a display device by the output unit 150 according to the present modified example and displayed by the display device. An output screen 71 illustrated in FIG. 7 includes an area 52, similarly to the output screen 51 illustrated in FIG. 5. Further, the output screen 71 includes an area 72 that displays a discharge destination prediction result and an area 73 that displays information relating to an item extracted by the extraction unit 140.

The area 72 displays a discharge destination indicated by a discharge destination prediction result. As described above, the discharge destination prediction result according to the present modified example includes a plurality of discharge destinations associated with a probability of a prediction, and therefore the area 72 displays a plurality of discharge destinations in which a probability of a prediction is indicated as a percent.

The area 73 displays information relating to an item extracted by the extraction unit 140, similarly to the area 54 illustrated in FIG. 5. The area 73 includes an area 74 that displays, in a selectable manner, any one of the plurality of discharge destinations included in the discharge destination prediction result. The output unit 150 displays, in the area 73, information of an item related to the discharge destination displayed in the area 74 among the items extracted by the extraction unit 140, similarly to the area 54. When the discharge destination displayed in the area 74 is changed, information of an item related to the changed discharge destination is displayed in the area 73.

Note that the output unit 150 may display all pieces of information relating to an item of each of the plurality of discharge destinations on the output screen 71 in association with the discharge destination with respect to each discharge destination. Further, when the discharge destination prediction result includes information of a prediction model used for a prediction and information representing a reason why a probability is calculated and the like, the output unit 150 may display these pieces of information. The acquiring unit 120 may acquire, based on these pieces of information, one or a plurality of prediction models from the model storage unit 110.

In this manner, the output unit 150 outputs the item related to each discharge destination in association with each of discharge destinations. Thereby, the medical information processing device 100 according to the present modified example can cause a medical practitioner to easily understand, even when a discharge destination prediction result includes a plurality of discharge destinations, by what item of an electronic medical record each predicted discharge destination is affected.

### Modified Example 2

The extraction unit 140 may accumulate, in a storage device or the like, an extraction result and a discharge destination prediction result in association with each other. The output unit 150 may output an output screen that displays the accumulated extraction result and discharge destination prediction result in a selectable manner.

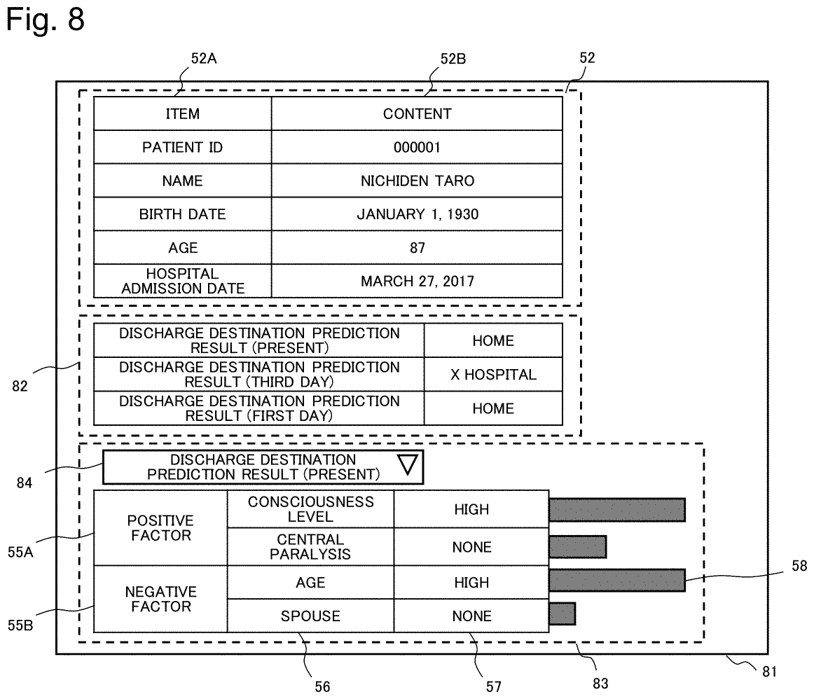

FIG. 8 is a diagram illustrating one example of an output screen output to a display device by the output unit 150 according to the present modified example and displayed by the display device. An output screen 81 illustrated in FIG. 8 includes an area 52, similarly to the output screen 51 illustrated in FIG. 5. Further, the output screen 81 includes an area 82 that displays a history of a discharge destination prediction result and an area 83 that displays information relating to an item extracted by the extraction unit 140.

The area 82 displays a history of a discharge destination indicated by a discharge destination prediction result. FIG. 8 includes a discharge destination prediction result of a first day, a discharge destination prediction result of a third day, and a discharge destination prediction result of a day (displayed as a present time) in which the output screen 81 is displayed, but may include a discharge destination prediction result of another day.

The area 83 displays information relating to an item extracted by the extraction unit 140, similarly to the area 54 illustrated in FIG. 5. The area 83 includes an area 84 that displays, in a selectable manner, information indicating a time at which a discharge destination is predicted. The output unit 150 displays, in the area 83, information of an item associated with a discharge destination prediction result at a time of being displayed in the area 84 among the items extracted by the extraction unit 140, similarly to the area 54. When the information displayed in the area 84 is changed, information of an item associated with the discharge destination prediction result at a time indicated by the changed information is displayed in the area 83.

Note that the output unit 150 may display, with respect to all histories of a discharge destination prediction result, information relating to an item associated with the discharge destination prediction result on the output screen 81.

In this manner, the output unit 150 outputs a history of a discharge destination prediction result, together with an item of an electronic medical record extracted from a prediction model 43 related to the discharge destination prediction result. Thereby, the medical information processing device 100 according to the present modified example can cause a medical practitioner to easily understand transition of a discharge destination indicated by a discharge destination prediction result and an item that affects a prediction of the discharge destination.

### Modified Example 3

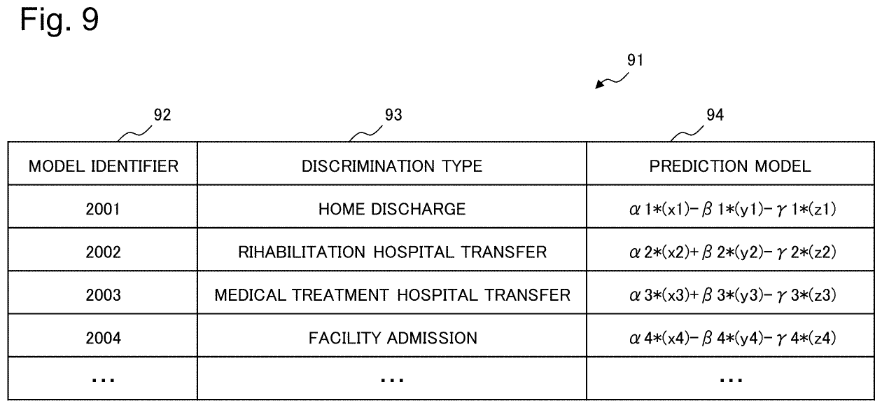

A prediction model included in model information stored in the model storage unit 110 may be able to predict one discharge destination by a calculated numerical value. FIG. 9 is a diagram illustrating one example of model information stored in the model storage unit 110 according to the present modified example. The model storage unit 110 stores model information 91, for example, as illustrated in FIG. 9. The model information 91 includes a model identifier 92 that identifies each prediction model, a discrimination type 93, and a prediction model 94. Note that the model information 91 may include information other than the information illustrated in FIG. 4. The model identifier 92 and the prediction model 94 are information similar to the model identifier 42 and the prediction model 43, respectively.

The determination type 93 indicates a type of an operation for one discharge destination discriminated by a prediction using each prediction model 94. For example, a determination type 93 in which the model identifier 92 is “2001” is “home discharge” being a type of an operation for a home. In a prediction using a prediction model 94 associated with the discrimination type 93, it can be predicted whether a discharge destination is a “home”. The prediction model 94 is, for example, a model for performing binary classification. The model information 91 includes prediction models 94 for the number of predicted discharge destinations (places).

According to the present modified example, it is assumed that a discharge destination prediction result acquired by the acquiring unit 120 includes a plurality of discharge destinations, similarly to the modified example 1. Each discharge destination is associated with a probability of a prediction. The acquiring unit 120 acquires a prediction model 94 related to each of the plurality of discharge destinations included in the discharge destination prediction result. When the plurality of discharge destinations are, for example, a “home”, a “rehabilitation hospital”, a “medical treatment hospital”, and a “facility”, the acquiring unit 120 acquires a prediction model 94 in which the model identifier 92 is associated with each of “2001”, “2002”, “2003, and “2004” from the model information 91 illustrated in FIG. 9.

The analysis unit 130 and the extraction unit 140 execute an operation similar to the above-described operation for each discharge destination and supplies, as an extracted result, an item extracted for each of the plurality of discharge destinations included in the discharge destination prediction result to the output unit 150, together with the discharge destination prediction result.

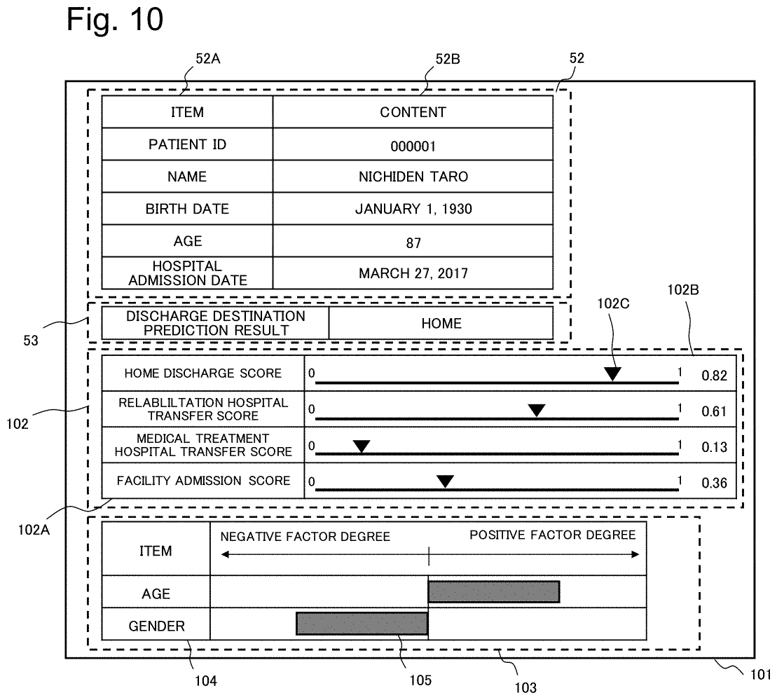

FIG. 10 is a diagram illustrating one example of an output screen output to a display device by the output unit 150 according to the present modified example and displayed by the display device. An output screen 101 illustrated in FIG. 10 includes an area 52 and an area 53, similarly to the output screen 51 illustrated in FIG. 5. Herein, a discharge destination displayed as a discharge destination prediction result included in the area 53 is a discharge destination in which the probability of the prediction is highest of the plurality of discharge destinations.

Further, the output screen 101 includes an area 102 that displays the probability of the prediction for the plurality of discharge destinations included in the discharge destination prediction result and an area 103 that displays information relating to an item extracted by the extraction unit 140.

The area 102 includes information 102A indicating to what discharge destination the displayed probability relates and probability information 102B indicating the probability. Herein, it is assumed that the probability illustrated in FIG. 10 is normalized to a number from 0 to 1.

In FIG. 10, for example, a “home discharge score” included in the information 102A indicates that the predicted discharge destination is a “home”, the operation for the discharge destination is “home discharge”, and the related probability information 102B is a probability when the discharge destination is predicted as a “home”.

The probability information 102B relating to the “home discharge score” may include a score (a numerical value from 0 to 1) or may include a graph in which a score is plotted on a bar representing a range from 0 to 1 and is expressed by a mark 102C, as illustrated in FIG. 10.

The area 53 displays the discharge destination indicated by the discharge destination prediction result.

The area 103 displays information relating to an item extracted by the extraction unit 140, similarly to the area 54 illustrated in FIG. 5. The area 103 displays information relating to an item of the discharge destination indicated in the area 53. The area 103 includes an item 104 and a parameter 105.

The item 104 is an item of an electronic medical record extracted by the extraction unit 140 from the prediction model 43. The parameter 105 is a parameter indicating, by using a graph, a total value of coefficients associated with the item 104. In FIG. 10, when the total value is positive, the total value is displayed by a bar graph extending in a right direction from a center, and when the total value is negative, the total value is displayed by a bar graph extending in a left direction from a center. In other words, the output unit 150 indicates that an “age” is an item (first item) contributing in such a way that the discharge destination is predicted as a “home” and a “gender” is an item (second item) contributing in such a way that the discharge destination is not predicted as a “home”.

Note that the output screen 101 may further display an item value.

In this manner, even when a prediction model included in model information stored in the model storage unit 110 can predict one discharge destination by a calculated numerical value, the medical information processing device 100 according to the present modified example can cause a medical practitioner to easily understand a discharge destination indicated by a discharge destination prediction result and an item that affects a prediction of a discharge destination.

### Modified Example 4

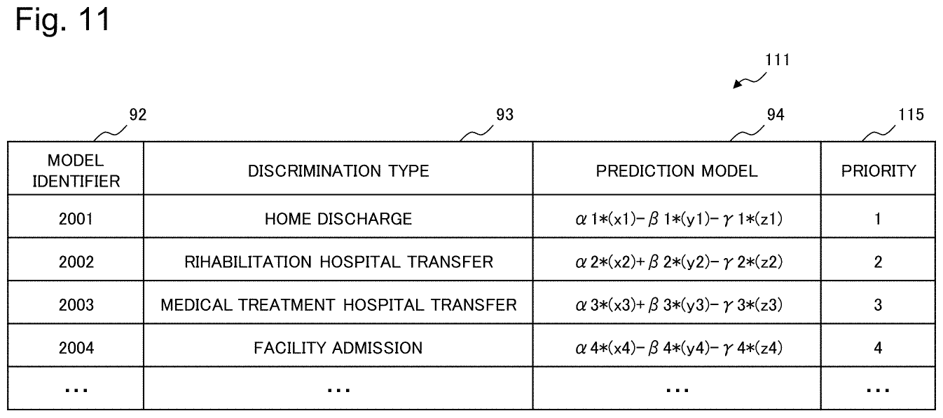

A prediction model included in model information stored in the model storage unit 110 may be able to predict one discharge destination in a predetermined order, by a calculated numerical value. FIG. 11 is a diagram illustrating one example of model information stored in the model storage unit 110 according to the present modified example. The model storage unit 110 stores model information 111, for example, as illustrated in FIG. 11. The model information 111 includes, similarly to the model information 91, a model identifier 92 that identifies each prediction model, a discrimination type 93, and a prediction model 94. The model information 111 further includes a priority 115.

The priority 115 indicates, when a prediction is performed by using the model information 111, an order of the prediction model 94 to be used. In other words, when a prediction is performed by using the model information 111, the priority 115 urges the prediction model 94 to be used for the prediction in ascending order from a small numerical value.

Herein, with regard to a patient of a mild case, a discharge destination is frequently predicted as a “home”. Further, as a severity increases, a place in which better facilities are arranged is frequently predicted as a discharge destination. Therefore, when the model information 111 is used for a prediction, the priority 115 is provided in such a way that a prediction model 94 related to a discharge destination in which a mild case is determined is used before a prediction model 94 in which a severe case is determined.

When a discharge destination indicated by a discharge destination prediction result indicates one discharge destination, for example, when the discharge destination is a “home”, the acquiring unit 120 acquires the prediction model 94 in which the model identifier 92 is associated with “2001” from the model information 111.

Further, when the discharge destination is, for example, a “medical treatment hospital”, the acquiring unit 120 acquires a prediction model 94 in which the model identifier 92 is associated with “2003” from the model information 111. At that time, the acquiring unit 120 may acquire a prediction model 94 in which the model identifier 92 is associated with each of “2001” and “2002”. In this case, for example, the analysis unit 130 or the extraction unit 140 may predict the above-described probability.

Further, when the discharge destination indicated by the discharge destination prediction result indicates a plurality of discharge destinations each associated with a probability, the acquiring unit 120 may acquire a prediction model 94 associated with each of the plurality of discharge destinations or may acquire a prediction model 94 associated with a discharge destination having a highest probability.

Even when such a prediction model 94 is used, the medical information processing device 100 according to the present example embodiment can output a discharge destination prediction result and an item of an electronic medical record extracted from a prediction model 94 related to the discharge destination prediction result in association with each other.

### Modified Example 5

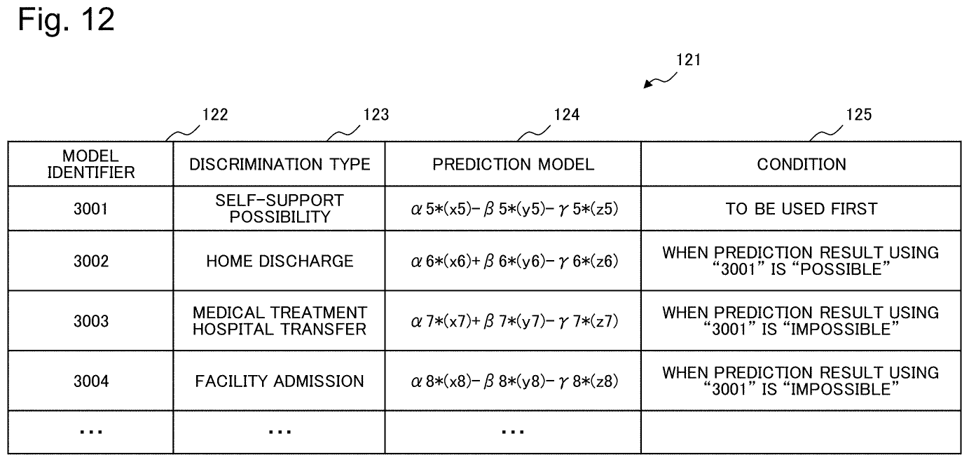

A prediction model included in model information stored in the model storage unit 110 may be generated in such a way that a different prediction model is used depending on a condition. FIG. 12 is a diagram illustrating one example of model information stored in the model storage unit 110 according to the present modified example. The model storage unit 110 stores model information 121, for example, as illustrated in FIG. 12. The model information 121 includes a model identifier 122 that identifies each prediction model, a discrimination type 123, a prediction model 124, and a condition 125.

The model identifier 122, the discrimination type 123, and the prediction model 124 correspond to the model identifier 92, the discrimination type 93, and the prediction model 94, respectively.

In the discrimination type 123, a “self-support possibility” indicates that an associated prediction model 124 is a model that discriminates whether to be self-supportable. In a case of being self-supportable, a discharge destination is frequently predicted as a “home” or a “rehabilitation hospital” for performing rehabilitation. Therefore, with regard to the prediction model 124 according to the present modified example, a prediction model 124 that determines whether to be self-supportable is included and first, a condition 125 being information that urges a prediction to be performed by using the prediction model 124 is associated with the prediction model 124.

The condition 125 indicates a condition when the prediction model 124 included in the model information 121 is used for predicting a discharge destination. When the prediction model 124 is used for predicting a discharge destination, a device used for the prediction performs the prediction by referring to the condition 125 and first using a prediction model 124 in which the model identifier 122 is “3001”. Thereafter, the device used for the prediction selects one or a plurality of prediction models 124 that satisfy the condition 125 and performs the prediction.

When the predicted discharge destination is a “home”, the acquiring unit 120 refers to the condition 125 and acquires, from the model information 121, a prediction model 124 in which the model identifier 122 is associated with “3001” and a prediction model 124 in which the model identifier 122 is associated with “3002”.

Even when such a prediction model 124 is used, the medical information processing device 100 according to the present example embodiment can output a discharge destination prediction result and an item of an electronic medical record extracted from a prediction model 124 related to the discharge destination prediction result in association with each other.

### Modified Example 6

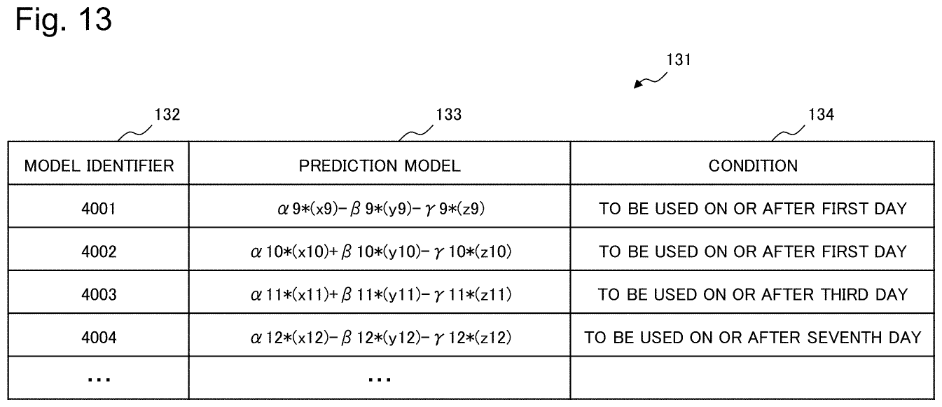

A prediction model included in model information stored in the model storage unit 110 may be associated with a condition different from the condition illustrated in FIG. 12. FIG. 13 is a diagram illustrating one example of model information stored in the model storage unit 110 according to the present modified example. The model storage unit 110 stores model information 131, for example, as illustrated in FIG. 13. The model information 131 includes a model identifier 132 that identifies each prediction model, a prediction model 133, and a condition 134. Note that the model information 131 may include the discrimination type 123 described above.

The model identifier 132 and the prediction model 133 correspond to the model identifier 92 and the prediction model 94, respectively.

The condition 134 indicates a condition when the prediction model 133 included in the model information 131 is used for predicting a discharge destination. When the prediction model 133 is used for predicting a discharge destination, a device used for the prediction refers to the condition 134.

The condition 134 indicates a condition for changing the number of prediction models 133 to be used depending on how many days so far a patient of a target for predicting a discharge destination has been hospitalized. In a case of a first day of hospitalization of a patient, for example, a device used for a prediction performs the prediction by using a prediction model 133 in which the model identifier 132 is “4001” and a prediction model 133 in which the model identifier 132 is “4002”. Further, in a case of a third day of hospitalization of a patient, for example, a device used for a prediction performs the prediction by using a prediction model 133 in which the model identifier 132 is “4001”, a prediction model 133 in which the model identifier 132 is “4002”, and a prediction model 133 in which the model identifier 132 is “4003”.

When the number of days after hospitalization increases, the number of items to be input to an electronic medical record and a degree of details of an item value generally increase. Therefore, as described according to the present modified example, a prediction model 133 depending on the number of days after hospitalization may be previously stored in the model storage unit 110. The medical information processing device 100 may be configured to acquire a prediction model 133 depending on the number of days after hospitalization.

The acquiring unit 120 acquires, based on a discharge destination prediction result, information about how many days so far a patient for whom a discharge destination is predicted has been hospitalized. The acquiring unit 120 refers to the condition 134 and acquires a prediction model 133 that satisfies the condition 134 from the model information 131.

Even when such a prediction model 133 is used, the medical information processing device 100 according to the present example embodiment can output a discharge destination prediction result and an item of an electronic medical record extracted from a prediction model 133 related to the discharge destination prediction result in association with each other.

### Modified Example 7

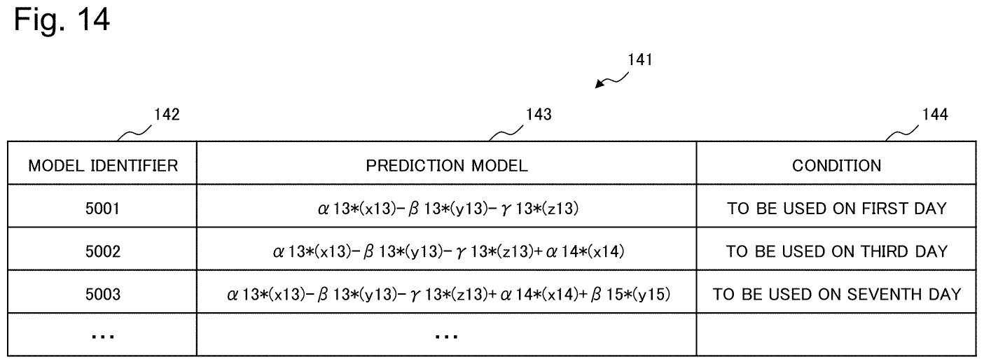

A prediction model included in model information stored in the model storage unit 110 may be associated with a condition different from the conditions as illustrated in FIG. 12 and FIG. 13. FIG. 14 is a diagram illustrating one example of model information stored in the model storage unit 110 according to the present modified example. The model storage unit 110 stores model information 141, for example, as illustrated in FIG. 14. The model information 141 includes a model identifier 142 that identifies each prediction model, a prediction model 143, and a condition 144. Note that the model information 141 may include the discrimination type 123 as described above.

The model identifier 142 and the prediction model 143 correspond to the model identifier 92 and the prediction model 94, respectively.

The condition 144 indicates a condition when the prediction model 143 included in the model information 141 is used for predicting a discharge destination. When the prediction model 143 is used for predicting a discharge destination, a device used for the prediction refers to the condition 144.

The condition 144 indicates a condition for changing a prediction model 133 to be used depending on how many days so far a patient of a target for predicting a discharge destination has been hospitalized. In a case of a first day of hospitalization of a patient, for example, a device used for the prediction performs the prediction by using a prediction model 143 in which the model identifier 142 is “5001”. Further, in a case of a third day of hospitalization of a patient, for example, a device used for the prediction performs the prediction by using a prediction model 143 in which the model identifier 142 is “5002”.

With regard to the prediction model 143, the number of items increases depending on the number of hospitalization days. In other words, with regard to the prediction model 143, the number of items being variables (explanatory variables) of an electronic medical record increases depending on the number of hospitalization days. When the number of days after hospitalization increases, the number of items to be input to an electronic medical record and a degree of details of an item value generally increase. Therefore, as described according to the present modified example, the prediction model 143 in which the number of items depending on the number of days after hospitalization increases may be previously stored in the model storage unit 110. The medical information processing device 100 may be configured to acquire a prediction model 143 in which the number of items increases depending on the number of days after hospitalization.

The acquiring unit 120 acquires, based on a discharge destination prediction result, information about how many days so far a patient for whom a discharge destination is predicted has been hospitalized. The acquiring unit 120 refers to the condition 144 and acquires a prediction model 143 that satisfies the condition 144 from the model information 141.

Even when such a prediction model 143 is used, the medical information processing device 100 according to the present example embodiment can output a discharge destination prediction result and an item of an electronic medical record extracted from a prediction model 143 related to the discharge destination prediction result in association with each other.

### Modified Example 8

The extraction unit 140 may accumulate, in a storage device or the like, an extraction result, a discharge destination prediction result, and information (e.g., a patient identifier (ID)) indicating a patient in association with one another. The output unit 150 may output an output screen, based on the accumulated information.

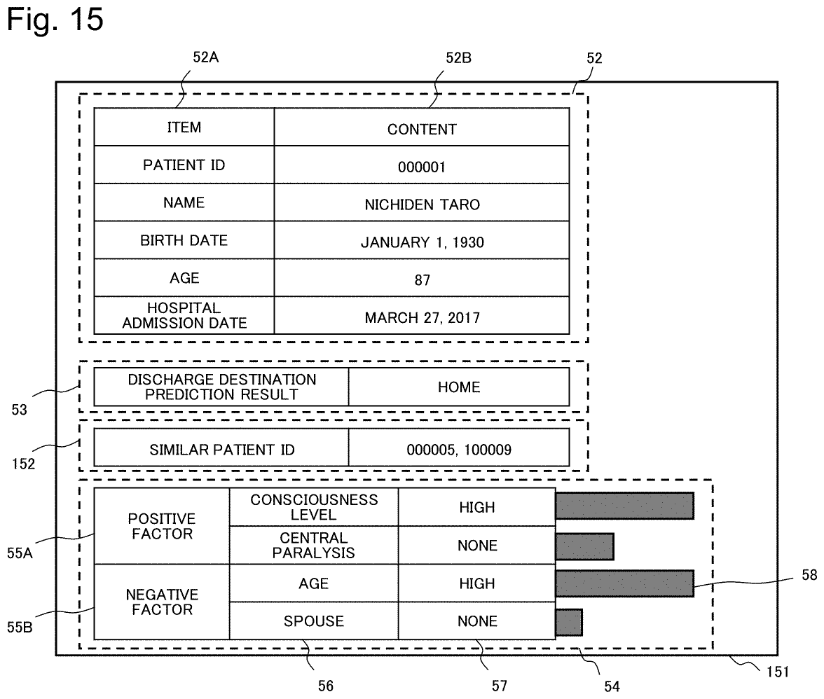

FIG. 15 is a diagram illustrating one example of an output screen output to a display device by the output unit 150 according to the present modified example and displayed by the display device. An output screen 151 illustrated in FIG. 15 includes an area 52, an area 53, and an area 54, similarly to the output screen 51 illustrated in FIG. 5. Further, the output screen 81 includes an area 152 that displays information of a patient having a similar discharge destination prediction result.

The output unit 150 refers to the accumulated information and identifies a patient having a similar extraction result and a similar discharge destination prediction result. The output unit 150 displays information indicating the identified patient in the area 152.

Thereby, a medical practitioner who refers to the output screen 151 can be caused to understand a similar patient having a similar extraction result and a similar discharge destination prediction result. Therefore, a medical practitioner can determine, by confirming a discharge destination of a similar patient displayed on the output screen 151, a discharge destination of a patient for whom a discharge destination is predicted. Therefore, the medical information processing device 100 according to the present modified example can suitably assist a medical practitioner in determining a highly reliable discharge destination.

Note that the output unit 150 in the medical information processing device 100 according to the present example embodiment may be configured, for example, to preferentially output an item weighted by a medical practitioner among items included in an extraction result. It is assumed that when a discharge destination indicated by a discharge destination prediction result is, for example, a “home”, the items included in the extraction result are an “age”, a “gender”, a “consciousness level”, and a “person living together”. Further, it is assumed that items weighted by a medical practitioner when a discharge destination is a “home” are a “consciousness level” and a “presence or absence of a complication”. In this case, the output unit 150 may output the “consciousness level” preferentially to the “age”, the “gender”, and the “person living together”.

### Third Example Embodiment

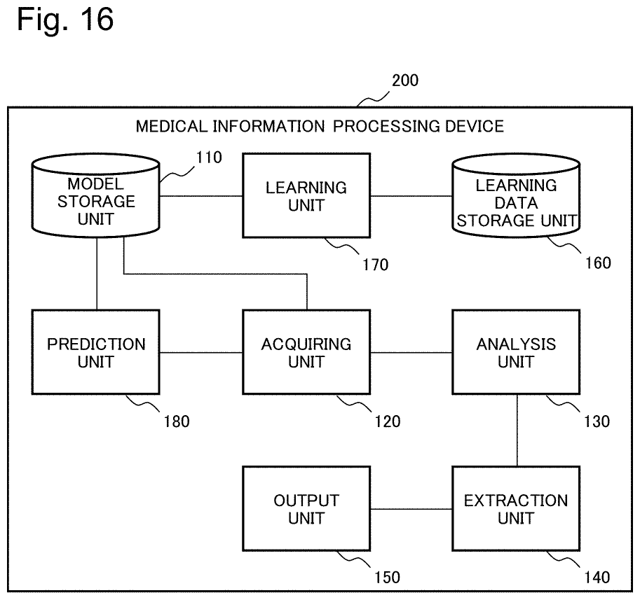

FIG. 16 is a block diagram illustrating one example of a configuration of a medical information processing device 200 according to a third example embodiment of the present disclosure. As illustrated in FIG. 16, the medical information processing device 200 includes a model storage unit 110, an acquiring unit 120, an analysis unit 130, an extraction unit 140, an output unit 150, a learning data storage unit 160, a learning unit 170, and a prediction unit 180. The medical information processing device 200 is configured by further including the learning data storage unit 160, the learning unit 170, and the prediction unit 180 for the medical information processing device 100.

The learning data storage unit 160 stores learning data. The learning data are an electronic medical record including, for example, information of a discharge destination. Note that a type and the number of pieces of learning data are not specifically limited. Note that the learning data storage unit 160 may be configured to be achieved by a storage device separate from the medical information processing device 200.

The learning unit 170 performs learning by using the learning data stored in the learning data storage unit 160 and generates a prediction model. The learning unit 170 may perform any machine learning. The learning unit 170 may perform, for example, heterogeneous mixture learning. Further, the learning unit 170 may perform, when an item of an electronic medical record is added with a weight, learning depending on the weight. The weight added to an item of an electronic medical record is a degree of importance added by a medical practitioner as an item that affects a discharge destination. For example, in a case of learning using an electronic medical record in which a discharge destination is a “home”, the weight is added, for example, to a “consciousness level” and a “presence or absence of a complication”. The learning unit 170 stores, as model information, a generated prediction model and an identifier that identifies the prediction model in the model storage unit 110 in association with each other.

The prediction unit 180 predicts a discharge destination by using the model information stored in the model storage unit 110. A method of the prediction performed by the prediction unit 180 is not specifically limited. The prediction unit 180 may perform a prediction according to the model information stored in the model storage unit 110. Further, the prediction unit 180 may predict a discharge destination according to a previously set condition.

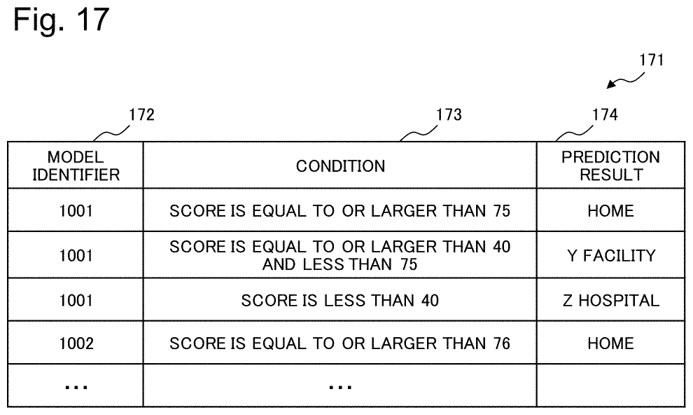

FIG. 17 is a diagram illustrating one example of condition information referred to when the prediction unit 180 performs a prediction. The condition information may be stored in the model storage unit 110 or may be stored in the prediction unit 180.

As illustrated in FIG. 17, condition information 171 includes a model identifier 172, a condition 173, and a prediction result 174. The model identifier 172 identifies a model. The model identifier 172 corresponds to, for example, the model identifier 42 in FIG. 4. The condition 173 indicates a condition to be satisfied by a result to be acquired when a prediction model is used. The prediction result 174 indicates a discharge destination to be output when a condition is satisfied.

It is assumed that the prediction unit 180 performs, when performing a prediction, for example, by using the model information 41 illustrated in FIG. 4, a prediction by using a prediction model 43 in which the model identifier 42 is “1001”. The prediction unit 180 identifies, by comparison with the condition 173, a condition satisfied by a value calculated by using the prediction model 43 in which the model identifier 42 is “1001” and outputs the prediction result 174 related to the identified condition 173 as a discharge destination prediction result.

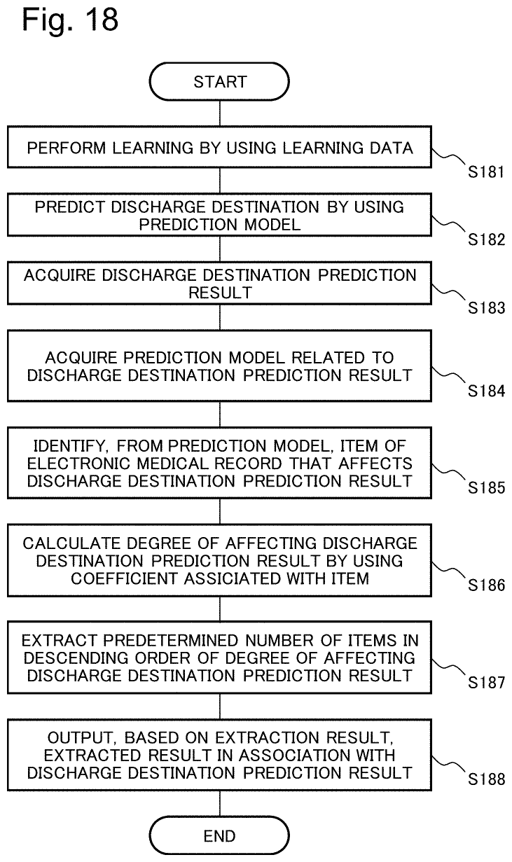

FIG. 18 is a flowchart illustrating one example of a flow of processing of the medical information processing device 200 according to the present example embodiment. As illustrated in FIG. 18, the learning unit 170 performs learning by using learning data (step S181). A prediction model as a result of performing the learning is stored in the model storage unit 110.

The prediction unit 180 predicts a discharge destination by using the prediction model stored in the model storage unit 110 (step S182). Note that a timing of executing step S182 may be a time at which an instruction for a prediction of a discharge destination is input, for example, by a medical practitioner or may be any time. A timing of executing step S182 may be a time at which an electronic medical record is input to the medical information processing device 200.

Thereafter, the medical information processing device 200 executes processing similar to step S61 to step S66. (step S183 to step S188).

As described above, the medical information processing device 200 terminates processing.

As described above, the medical information processing device 200 according to the present example embodiment includes the learning data storage unit 160, the learning unit 170, and the prediction unit 180, in addition to the medical information processing device 100. Thereby, the acquiring unit 120 can acquire, from the model storage unit 110, a prediction model used for predicting a discharge destination, and therefore the extraction unit 140 can accurately extract an item that affects the prediction of the discharge destination. Therefore, the medical information processing device 200 according to the present example embodiment can more suitably assist a medical practitioner in determining a highly reliable discharge destination.

(Regarding a Hardware Configuration)

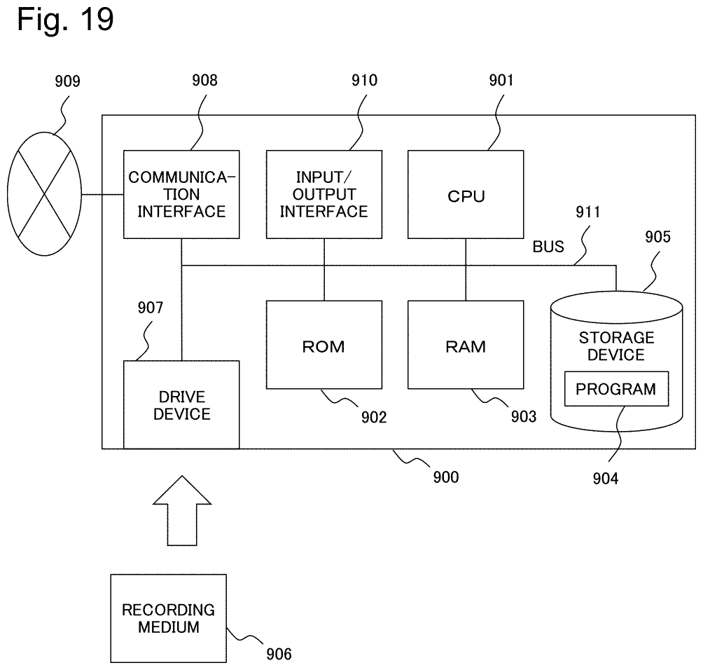

According to example embodiments of the present disclosure, each component of a medical information processing device (10, 100, and 200) indicates a block of a function unit. Some or all of the components of each device are achieved by any combination of an information processing device 900, for example, as illustrated in FIG. 19 and a program. FIG. 19 is a block diagram illustrating one example of a hardware configuration of the information processing device 900 that achieves the components of each device. The information processing device 900 includes the following configuration as one example.
A central processing unit (CPU) 901
A read only memory (ROM) 902
A random access memory (RAM) 903
A program 904 loaded on the RAM 903
A storage device 905 storing the program 904
A drive device 907 executing read/write to/from a recording medium 906
A communication interface 908 for connection to a communication network 909
An input/output interface 910 executing input/output of data
A Bus 911 connecting the components

The components of the medical information processing device (10, 100, and 200) according to the example embodiments are achieved by acquiring and executing the program 904 achieving functions thereof by using the CPU 901. The program 904 achieving functions of the components of the medical information processing device (10, 100, and 200) is previously stored, for example, in the storage device 905 or on the ROM 902, and is loaded onto the RAM 903 by the CPU 901 and executed, as necessary. Note that the program 904 may be supplied to the CPU 901 via the communication network 909 or may be previously stored on the recording medium 906 and supplied to the CPU 901 by reading the program by using the drive device 907.

A method of achieving the medical information processing device (10, 100, and 200) includes various modified examples. The medical information processing device (10, 100, and 200) may be achieved by any combination of the information processing device 900 and a program that are separately provided for each component. Further, a plurality of components included in the medical information processing device (10, 100, ad 200) may be achieved by any combination of one information processing device 900 and a program.

Further, some or all of the components of the medical information processing device (10, 100, and 200) are achieved by another general-purpose or dedicated circuit, a processor, or a combination thereof. These may be configured by a single chip or may be configured by a plurality of chips connected via a bus.

Some or all of the components of the medical information processing device (10, 100, and 200) may be achieved by a combination of the circuit descried above and a program.

When some or all of the components of the medical information processing device (10, 100, and 200) are achieved by a plurality of information processing devices, a circuit, and the like, a plurality of information processing devices, a circuit, and the like may be disposed in a centralized manner or in a distributed manner. An information processing device, a circuit, and the like may be achieved, for example, as a form such as a client and server system, a cloud computing system, and the like in which these are connected via a communication network.

While the invention has been particularly shown and described with reference to exemplary example embodiments thereof, the invention is not limited to these example embodiments. It will be understood by those of ordinary skill in the art that various changes in form and details may be made therein without departing from the spirit and scope of the present invention as defined by the claims.

This application is based upon and claims the benefit of priority from Japanese patent application No. 2017-165378, filed on Aug. 30, 2017, the disclosure of which is incorporated herein in its entirety by reference.

### REFERENCE SIGNS LIST

10 Medical information processing device
11 Acquiring unit
12 Extraction unit
13 Output unit
100 Medical information processing device
110 Model storage unit
120 Acquiring unit
130 Analysis unit
140 Extraction unit
150 Output unit
160 Learning data storage unit
170 Learning unit
180 Prediction unit
200 Medical information processing device

1. A medical information processing device comprising:
at least one memory storing instructions; and
at least one processor configured to access the at least one memory and execute the instructions to:
acquire a discharge destination prediction result and a prediction model related to the discharge destination prediction result, wherein the discharge destination prediction result is a result of predicting a discharge destination and wherein the model is used for classifying the discharge destination by using a data item included in an electronic medical record as an explanatory variable;
extract, from the prediction model, the data item that affects a prediction of the discharge destination; and
output, based on an extraction result, the data item in association with the discharge destination prediction result.

2. The medical information processing device according to claim 1, wherein
the at least one processor is further configured to execute the instructions to:
output the data item and an item value of the data item in association with each other.

3. The medical information processing device according to claim 1, wherein
the at least one processor is further configured to execute the instructions to:
extract, when related information associated with the data item satisfies a predetermined condition, the data item from the prediction model, and
output the related information in association with the data item.

4. The medical information processing device according to claim 3, wherein
the at least one processor is further configured to execute the instructions to:
extract, based on a sign of the related information, a first item that contributes to a prediction as the discharge destination and a second item that contributes to a prediction as another discharge destination different from the discharge destination among the data item, and
output the first item and first information representing that contribution is made to a prediction as the discharge destination in association with each other, and outputs the second item in association with second information different from the first information.

5. The medical information processing device according to claim 1, wherein
the discharge destination prediction result includes a plurality of discharge destinations and a probability of a prediction for each discharge destination, and
the at least one processor is further configured to execute the instructions to:
extract, from the prediction model, the data item for the discharge destination, and
output, for each of the discharge destinations, the data item related to the discharge destination in association with the discharge destination prediction result.

6. The medical information processing device according to claim 1, wherein
the at least one processor is further configured to execute the instructions to:
output a history of the discharge destination prediction result, together with the data item related to the discharge destination prediction result.

7. A medical information processing method comprising:
acquiring a discharge destination prediction result and a prediction model related to the discharge destination prediction result, wherein the discharge destination prediction result is a result of predicting a discharge destination and the prediction model is used for classifying the discharge destination by using a data item included in an electronic medical record as an explanatory variable;
extracting, from the prediction model, the data item that affects a prediction of the discharge destination; and
outputting, based on an extraction result, the data item in association with the discharge destination prediction result.

8. The medical information processing method according to claim 7, further comprising:
outputting the data item and an item value of the data item in association with each other.

9. A non-transitory computer-readable storage medium storing a computer program that causes a computer to execute the processes of:
acquiring a discharge destination prediction result and a prediction model related to the discharge destination prediction result, wherein the discharge destination prediction result is a result of predicting a discharge destination and wherein the prediction model is used for classifying the discharge destination by using a data item included in an electronic medical record as an explanatory variable;
extracting, from the prediction model, the data item that affects a prediction of the discharge destination; and
outputting, based on an extraction result, the data item in association with the discharge destination prediction result.

10. The non-transitory computer-readable storage medium according to claim 9, wherein
the computer program further causes the computer to execute the processes of:
outputting the data item and an item value of the data item in association with each other.

11. The medical information processing method according to claim 7, further comprising:
extracting, when related information associated with the data item satisfies the predetermined condition, the data item from the prediction model, and
outputting the related information in association with the data item.

12. The medical information processing method according to claim 11, further comprising:
extracting, based on a sign of the related information, a first item that contributes to a prediction as the discharge destination and a second item that contributes to a prediction as another discharge destination different from the discharge destination among the data item, and
outputting the first item and first information representing that contribution is made to a prediction as the discharge destination in association with each other, and outputs the second item in association with second information different from the first information.

13. The medical information processing method according to claim 7, further comprising:
extracting, from the prediction model, the data item for the discharge destination, and
outputting, for each of the discharge destinations, the data item related to the discharge destination in association with the discharge destination prediction result, the discharge destination prediction result including a plurality of discharge destinations and a probability of a prediction for each discharge destination.

14. The medical information processing method according to claim 7, further comprising:
outputting a history of the discharge destination prediction result, together with the data item related to the discharge destination prediction result.
